(19) United States
(12) Patent Application Publication
(10) Pub. No.: US 2008/0057033 A1
(43) Pub. Date: Mar. 6, 2008

(54) Neural Stem Cells and Methods of Generating and Utilizing Same

(76) Inventors: Peter Lonai, Rehovot (IL); Rozmari Stirbu-Lonai, Rehovot (IL)

Correspondence Address: Martin D Moynihan;PRTSI Inc, P O Box 16446, Arlington, VA, 22215, US

(21) Appl. No.: 10/559,929

(22) PCT Filed: Jun. 9, 2004
(86) PCT No.: PCT/IL04/00489

§ 371 Date: May 2, 2007

Related U.S. Application Data
(60) Provisional application No. 60/477,364, filed on Jun. 11, 2003.

(51) Int. Cl. A61K 38/00; C07H 21/04; C07K 16/00; C12N 5/00; C12N 5/04

(52) U.S. Cl. 424/93.2; 435/366; 435/455; 530/350; 536/23.5

(57) ABSTRACT

Methods of generating neural stem cells and neural stem cell culture generated by such methods are provided.

### FIELD AND BACKGROUND OF THE INVENTION

The present invention relates to neural stem cells and to methods of generating same. The present invention further relates to method of utilizing such neural stem cells in treating neurological disorders characterized by loss or degeneration of neural cells.

Numerous diseases of the central nervous system (CNS) are either caused by, or lead to, loss of neural tissue. Since mature neurons do not proliferate, efficient treatment of such diseases necessitates neural cell transplantation.

One example of a disease characterized by loss of neural tissue is Parkinson's disease in which death of DOPA-ergic neurons leads to severe loss of voluntary movement and subsequent deterioration in the quality of life of an afflicted individual. Several studies have demonstrated that experimental Parkinsonism responds to locally injected embryonic CNS cells (Bjorklund and Lindvall Nat Neurosci 3, 537-544; 2000) proving that neural cell transplantation can be an effective therapeutic approach for this disease and providing the basis for using neural cells in treatment of other neuro-disorders such as Huntington chorea, Alheimer's disease, brain hemorrhage and myelination disorders (Bjorklund and Lindvall Ibid).

Since both practical and ethical arguments militate against using human embryonic material as a direct source of neural cell precursors, researchers have concentrated their efforts towards finding appropriate substitutes. Cloned stem cell cultures, free of elements causing graft versus host reaction and capable of colonizing specific areas of the CNS are regarded as one potential source of cells which can be used in neurological therapy.

Recent developments in stem cell technology have enabled the isolation of neural stem cells from embryonic stem (ES) cell cultures. Neural stem cell are pluripotent precursor cells which can be maintained in a proliferative undifferentiated state in culture and can be induced to differentiate into neurons, astrocytes and oligodendrocytes. Neural stem cells derived from ES cell cultures, are advantageous in therapy of neuro-disorders since it has been demonstrated that such cells can be successfully implanted in the CNS and since these cells are less likely to invoke an immune response when implanted in the body.

Methods of isolating neural stem from murine (Okabe et al. Mech Dev 59, 89-102. 1996) and human ES cells (Zhang et al. Nat Biotechnol 19, 1129-1133. 2001 and Reubinoff et al. Nat Biotechnol 19, 1134-1140. 2001) have been previously described. These and other similar methods are based on culture conditions, which induce the formation of ES cell aggregates, which develop into so-called embryoid bodies (Martin et al. Dev. Biol. 61. 230. 1977). In serum free defined media (Rizzino & Crowley Proc. Natl. Acad. Sci. USA 77. 457. 1980; Okabe et al. Mech. Dev. 59. 89. 1996) most non-neural elements of the embryoid body die and round cell aggregates, which are termed “neurospheres” appear. Neurospheres contain neural stem cells, which can be induced to differentiate into neurons and glia cells either in vitro or in vivo.

Although the neurosphere approach has been successfully used to isolate neural stem cells from ES cultures, methods utilizing this isolation step are typically difficult and time consuming to perform, are limited by a low yield of neural stem cells and poor reproducibility while the neural stem cells recovered using such methods are not clonal and thus do not ultimately lead to the generation of high quantities of neural stem cells.

Thus, there is a widely recognized need for methodology which can be used to generate large quantities of neural stem cells which can be used in cell replacement therapy.

While reducing the present invention to practice, the present inventor has formulated a novel approach for generating neural stem cells from embryonic stem cells. As is illustrated in the Examples section hereinbelow, the present methodology enables the generation of large quantities of neural cell precursors which when administered into the body target the CNS and are capable of developing into neurons and glial cells.

### SUMMARY OF THE INVENTION

According to one aspect of the present invention there is provided a stem cell culture comprising stem cells transformed to express GATA6 or GATA4 or active portions or modificants thereof.

According to another aspect of the present invention there is provided a stem cell expressing GATA6 or GATA4 or active portions or modificants thereof.

According to further features in preferred embodiments of the invention described below, the stem cells are embryonic stem cells.

According to still further features in the described preferred embodiments the embryonic stem cells are derived from an inner cell mass (ICM) layer of an embryo.

According to still further features in the described preferred embodiments the embryo is a human embryo.

According to still further features in the described preferred embodiments the stem cell is an early neural stem cell characterized by expression of at least one protein selected from the group consisting of nestin, class III β-tubulin, neural specific enolase, S-100, glial specific acidic fibrillary protein (GFAP).

According to still further features in the described preferred embodiments the stem cell is characterized by a neuron-like morphology.

According to yet another aspect of the present invention there is provided a nucleic acid construct comprising a polynucleotide sequence encoding a GATA6 or a GATA4 or active portions or modificants thereof.

According to still further features in the described preferred embodiments the nucleic acid construct further comprising at least one promoter sequence being for directing transcription of the polynucleotide sequence in stem cells.

According to still further features in the described preferred embodiments the at least one promoter sequence is not active in differentiated cells.

According to still further features in the described preferred embodiments the at least one promoter is selected from the group consisting of Spi2A minimal promoter and the hTERT promoter.

According to an additional aspect of the present invention there is provided a feeder cell population including feeder cells transformed to express GATA6 or GATA4 or active portions or modificants thereof.

According to still further features in the described preferred embodiments the feeder cells are fibroblasts.

According to still further features in the described preferred embodiments the feeder cells comprise a nuclei acid construct including a polynucleotide sequence encoding the GATA6 or the GATA4 or active portions or modificants thereof.

According to yet an additional aspect of the present invention there is provided a composition-of-matter comprising GATA6 or GATA4 or active portions or modificants thereof associated with a carrier suitable for directing intracellular delivery of the GATA6 or the GATA4 or active portions or modificants thereof.

According to still further features in the described preferred embodiments the carrier is a lipid carrier.

According to still further features in the described preferred embodiments the carrier is a protein carrier.

According to still another aspect of the present invention there is provided a method of generating neural stem cells comprising exposing stem cells to GATA6 or GATA4 or active portions or modificants thereof thereby generating neural stem cells.

According to still an additional aspect of the present invention there is provided a method of treating a neurological disorder characterized by neural cell degeneration or loss, the method comprising: (a) administering stem cells to a subject diagnosed with the neurological disorder; and (b) prior to, concomitant with or following administration, exposing the stem cells to GATA6 or GATA4 or active portions or modificants thereof to thereby treat the neurological disorder characterized by neural cell degeneration or loss.

According to further still an additional aspect of the present invention there is provided a method of treating a neurological disorder characterized by neural cell degeneration or loss, the method comprising: (a) expressing within stem cells GATA6 or GATA4 or active portions or modificants thereof to thereby generate neural stem cells; and (b) administering the neural stem cells to a subject diagnosed with the neurological disorder thereby treating the neurological disorder characterized by neural cell degeneration or loss.

According to still further features in the described preferred embodiments the exposing is effected for a time period sufficient for inducing expression in the stem cells of at least one protein selected from the group consisting of nestin, class III β-tubulin, neural specific enolase, S-100, glial specific acidic fibrillary protein (GFAP).

According to still further features in the described preferred embodiments the exposing is effected by transforming the stem cells with a nucleic acid construct capable of expressing the GATA6 or the GATA4 or active portions or modificants thereof in the stem cells.

According to still further features in the described preferred embodiments the exposing is effected by culturing the stem cells on a feeder cell layer expressing the GATA6 or the GATA4 or active portions or modificants thereof.

According to still further features in the described preferred embodiments the feeder cell layer is capable of secreting the GATA6 or the GATA4 or active portions or modificants thereof expressed thereby.

According to still further features in the described preferred embodiments the stem cells are cultured in a medium including DMEM/F12 with 5 μg/ml insulin, 100 μg/ml transferrin, 16.1 μg/ml putrescine, 5.2 ng/ml selenite and 6.3 ng/ml progesterone.

According to still further features in the described preferred embodiments the exposing is effected by culturing the stem cells in a culture medium supplemented with the GATA6 or the GATA4 or active portions or modificants thereof.

The present invention successfully addresses the shortcomings of the presently known configurations by providing a an efficient and easily applicable method of generating neural stem cells which are clonal and are capable of being utilized in cell replacement therapy.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

Terms:

As used herein the phrase “stem cells” refers to undifferentiated pluripotent cells which can be maintained in an undifferentiated, proliferative state in cell culture and are capable, under appropriate conditions, of differentiating into various cell types. The phrase “stem cells” refers to both adult stem cells which are recovered from adult tissues and to embryonic stem cells which are typically derived from the inner cell mass of a blastocyst.

As used herein the phrases “neural derivatives” or “neural cells” refers to cells characterized by thin extensions longer than four cell diameter, which typically stain with TuJ-1 anti tubulin III.

As used herein the phrases “neural cells” refers to cells which form the CNS and thus include astrocytes, neurons and glial cells.

As used herein the phrase “neural stem cell” refers to a pluripotent cell which has partially differentiated down the neural cell lineage pathway and thus displays some attributes (e.g., marker expression) of a neural cell. Such a cell is also referred to herein as an early neural stem (ENS) cell. A neural stem cell can be maintained in a proliferative undifferentiated state in culture and can be induced to differentiate into neurons, astrocytes and oligodendrocytes.

As used herein the phrase “active portion” when used in context with GATA6 or GATA4 refers to any portion of these proteins which is capable of regulating the transcription of its target genes. Such a portion can include the Zinc Finger domain coupled to the polymerase binding domain (Molkentin J. Biol. Chem. 275. 38949 2000).

As used herein the phrase “active modificant” when used in context with GATA6 or GATA4 refers to an allele, isoform or mutant of GATA6 or GATA4 which displays at least some of the activity attributed to GATA6 or GATA4 but includes a modified amino acid sequences which is a result of one or more alterations in the coding sequences thereof.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a three dimensional bar graph illustrating expression of FGF, FGFR and GATA genes in dominant negative mutant (dnFGFR) and wild type (Wt) embryoid bodies. Affimetrix DNA microarray analysis was conducted on day 0 (ES cells) and day 4 (EB) cultures. Note that Fgf4 (a member of a group of stem cell specific genes which is inhibited by differentiation), Fgfr2 and GATA6 are expressed in the undifferentiated ES cell.

FIGS. 2A-F illustrate detection of non-labeled wild type cells and LacZ labeled mutant cells in mixed cell cultures, demonstrating that ES cells can rescue differentiation of dnFGFR mutant ES cells. The EBs were grown for six days from individual cell lines (FIGS. 2A-C) or mixed ES cell cultures (FIGS. 2D-F). FIG. 2A—β-galactosidase positive wild type ROSA11 cells; FIG. 2B—β-galactosidase negative, AB2.2 wild type cells; FIG. 2C—1C3, a ROSA 11 derived, dominant negative mutant clone; FIG. 2D—control chimera made of ROSA 11 and AB2.2 cells; FIG. 2E-experimental chimera grown from mutant 1C3 and wild type AB2.2 cells. Note that in mixed cultures mutant cells colonize both cell layers of the EB and its fragments are localized in the central cavity. FIG. 2F—Fused embryoid body from a culture of mixed (ROSA11 and 1C3) aggregates; some or no rescue of the dominant negative mutant could be observed in this experiment suggesting that mutant rescue occurs over a limited radium. Size bars −25 micron.

FIG. 3 is a Northern blot illustrating that dnFgfr2 mutant ES cells and embryoid bodies do not express isotypes of Laminin-1 and Collagen type IV.

FIG. 4 illustrates that externally added mixed BM proteins (Matrigel—middle panel; purified laminin-1—right panel) rescues embryoid body differentiation of dnFgfr2 ES cells. Left panel shows an untreated dnFgfr2 mutant ES cell.

FIG. 5 is a Western blot illustrating that ES cells transformed by GATA4 or GATA6 express large amounts of laminin-1 (top panel) and collagen type IV (bottom panel) isotypes. Upper band identified by anti laminin-1 is the 400 kD α1 chain, the lower band is the ˜200 kD β1 and γ1 chain. Anti-collagen IV identifies the 210 kD α1 and β1 chains.

FIGS. 6A-D illustrate that GATA4 transformed ES cells rescue epiblast differentiation directed by the dnFgfr2 mutant. FIG. 6A—wild type embryoid bodies stained with neutral red; FIG. 6B—GATA4 transformed wild type ES cells (neutral red staining) form bubble-like aggregates containing BM proteins; FIG. 6C—dnFgfr2-mutant ES cells stained for β-galactosidase and counterstained with neutral red. Note lack of differentiation as compared to FIG. 6A; FIG. 6C—mixed culture of dnFgfr mutant and GATA4-transformed wild type cells; note β-galactosidase positive mutant-derived epiblast in the middle, surrounding an apoptotic central cavity, also note the surrounding GATA4 transformed cells.

FIGS. 7A-B illustrate that the morphology of GATA6 transfected RSA11 ES cell clones. The cells were co-transfected by pCAGI-GATA6 and a puromycine resistance gene. Following puromycine selection, individual clones of small shiny cells appear, with neuron-like cells around their periphery.

FIGS. 8a-f illustrate that GATA6 transformed ES cells and their neurofibers stain for neuron specific tubulin III. Left panel—phase contrast, right panel—tubulin-Cy3.

FIGS. 9a-f illustrate staining of GATA6 transformed neuron-like ES cell derivatives with neural specific antibodies.

FIGS. 10A-D illustrate homing of GATA6 transfected neuron-like cells in the brain. 5×10⁴ β-gal positive cells were injected into the lateral ventricles of newborn mice. 35 days later the cells were detected in cryostate sections by LacZ staining.

FIG. 10A illustrates a low magnification view of the hippocampus; arrows show labeled cells. FIGS. 10B and C illustrate higher magnification views of a similar location. FIG. 10D illustrates colonization in an area close to the C3 area of the hippocampus. Arrow in FIGS. 10B and D show cells with neural extensions.

FIG. 11A-C illustrates that GATA6 transfected neuron-like cells participate in neural development. 10-15 cells were injected into C57B1/6 blastocysts, which were transplanted into the uterus of pseudpregnant females. The embryos were isolated at mid-gestation (E11.5) and were stained for β-gal. Arrows show derivatives of the transplanted cells in the hind-, mid- and forebrain. FIG. 11A illustrates an E1.5 chimera; note that GATA6 transfected neural precursors home to sites in the CNS. FIG. 11B illustrates the head of another E11.5 chimeric embryo; arrows show derivatives of GATA-6 transformed cells in the fore-, mid- and hindbrain and in neural cells of the otic cyst; the arrowhead shows β-gal staining in the umbilical cord, which is endoderm derived. FIG. 11C illustrates that cells localized in the floor plate region extending from the midbrain to the caudal extreme of the spinal cord. Black arrow—midbrain-hindbrain junction, white arrow—floor plate at the tail tip.

### DESCRIPTION OF THE PREFERRED EMBODIMENTS

The present invention is of neural stem cells and methods of generating and utilizing same. Specifically, the methodology of the present invention can be used to generate large quantities of neural stem cells which can be cloned and efficiently used in various cell replacement therapeutic approaches.

The principles and operation of the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Advances in stem cell technology now enable the generation and utilization of neuronal cell precursors which can potentially be used in cell replacement therapy. However, due to limitations inherent to such methodologies, the quality and in particular the quantity of neural stem cells currently generated from embryonic stem cell cultures is insufficient for practical therapeutic applications.

The present invention provides a novel approach for isolating early neural stem (ENS) cells. The present approach utilizes GATA6 or GATA4, known regulators of endoderm development, to induce pre-neuronal differentiation in ES cells. As is illustrated in the Examples section hereinbelow, neural stem cells generated according to the teachings of the present invention specifically target and colonize the CNS. In contrast to prior art methods which utilize the neurosphere approach, the present methodology can be utilized to easily and rapidly generate large quantities of transplantation competent neural stem cells in a highly reproducible manner.

Thus according to one aspect of the present invention there is provided a method of generating neural stem cells which can be used in cell replacement therapy.

The method according to this aspect of the present invention is effected by comprising exposing stem cells to GATA6 or GATA4 or active portions or modificants thereof thereby generating neural stem cells.

Stem cells utilized by the present invention are mammalian stem cells preferably of a human origin.

The stem cells can also be obtained using well-known cell-culture methods. For example, human embryonic stem cells can be isolated from human blastocysts. Human blastocysts are typically obtained from human in vivo preimplantation embryos or from in vitro fertilized (IVF) embryos. Alternatively, a single cell human embryo can be expanded to the blastocyst stage. For the isolation of human ES cells the zona pellucida is removed from the blastocyst and the inner cell mass (ICM) is isolated by immunosurgery, in which the trophectoderm cells are lysed and removed from the intact ICM by gentle pipetting. The ICM is then plated in a tissue culture flask containing the appropriate medium which enables its outgrowth. Following 9 to 15 days, the ICM derived outgrowth is dissociated into clumps either by a mechanical dissociation or by an enzymatic degradation and the cells are then re-plated on a fresh tissue culture medium. Colonies demonstrating undifferentiated morphology are individually selected by micropipette, mechanically dissociated into clumps, and re-plated. Resulting ES cells are then routinely split every 1-2 weeks. For further details on methods of preparation human ES cells see Thomson et al., [U.S. Pat. No. 5,843,780; Science 282: 1145, 1998; Curr. Top. Dev. Biol. 38: 133, 1998; Proc. Natl. Acad. Sci. USA 92: 7844, 1995]; Bongso et al., [Hum Reprod 4: 706, 1989]; Gardner et al., [Fertil. Steril. 69: 84, 1998].

It will be appreciated that commercially available stem cells can be also be used with this aspect of the present invention. Human ES cells can be purchased from the NIH human embryonic stem cells registry (http://escr.nih.gov). Non-limiting examples of commercially available embryonic stem cell lines are BG01, BG02, BG03, BG04, CY12, CY30, CY92, CY10, TE03 and TE32.

Stem cells used by the present invention can be also derived from human embryonic germ (EG) cells. Human EG cells are prepared from the primordial germ cells obtained from human fetuses of about 8-11 weeks of gestation using laboratory techniques known to anyone skilled in the arts. The genital ridges are dissociated and cut into small chunks which are thereafter disaggregated into cells by mechanical dissociation. The EG cells are then grown in tissue culture flasks with the appropriate medium. The cells are cultured with daily replacement of medium until a cell morphology consistent with EG cells is observed, typically after 7-30 days or 1-4 passages. For additional details on methods of preparation human EG cells see Shamblott et al. [Proc. Natl. Acad. Sci. USA 95: 13726, 1998] and U.S. Pat. No. 6,090,622.

It will be appreciated that ES cells can be obtained from other species as well, including mouse Mills and Bradley, 2001), golden hamster [Doetschman et al., 1988, Dev Biol. 127: 224-7], rat [Iannaccone et al., 1994, Dev Biol. 163: 288-92] rabbit [Giles et al. 1993, Mol Reprod Dev. 36: 130-8; Graves and Moreadith, 1993, Mol Reprod Dev. 1993, 36: 424-33], several domestic animal species Notarianni et al., 1991, J Reprod Fertil Suppl. 43: 255-60; Wheeler 1994, Reprod Fertil Dev. 6: 563-8; Mitalipova et al., 2001, Cloning. 3: 59-67] and non-human primate species (Rhesus monkey and marmoset) Thomson et al., 1995, Proc Natl Acad Sci USA. 92: 7844-8; Thomson et al., 1996, Biol Reprod. 55: 254-9].

It will be appreciated that in order to maintain ES cells in an undifferentiated state, ES culture must be supplemented with factors which maintain cell proliferation, inhibit ES cell differentiation and preserve pluripotency. Current methods for culturing ES cells include the use of human or mouse feeder cells, serum free medium or conditioned medium.

The most common method for culturing ES cells is based on mouse embryonic fibroblasts (MEF) as a feeder cell layer supplemented with tissue culture medium containing serum or leukemia inhibitor factor (LIF) which supports the proliferation and the pluripotency of the ES cells (Thomson et al, 1998; Reubinoff et al 2000). MEF cells are derived from day 12-13 mouse embryos in medium supplemented with fetal bovine serum. Under these conditions ES cells can be maintained for many passages in culture while preserving their phenotype and functional characteristics.

ES cells can also be cultured on MEF under serum-free conditions using serum replacement supplemented with basic fibroblast growth factor (bFGF) (Amit et al. Dev. Biol. 227: 271-8). Under these conditions the cloning efficiency of ES cells is 4 times higher than under fetal bovine serum. In addition, following 6 months of culturing under serum replacement the ES cells still maintain their pluripotency as indicated by their ability to form teratomas which contain all three embryonic germ layers.

ES cells can also be cultured in a feeder-free environment. Stem cells are grown on a solid surface such as an extracellular matrix (e.g., Matrigel® or laminin) in the presence of culture medium. The culture medium used for growing the stem cells contains factors that effectively inhibit differentiation and promote their growth such as MEF-conditioned medium and bFGF.

The stem cells can also be adult stem cells recovered from adult tissues including the CNS (Tissue Engineering vol 8, p 739; 2002 and Ballas et al. J Cell Biochem Suppl. 2002; 38:20-8). Since such cells have shown to be pluripotent, responsive to various factors which affect embryonic stem cells and capable of differentiating into various cell types [Passier et al. Cardiovasc Res. 2003 May 1;58(2):324-35] it is conceivable that the method of the present invention can also utilize such cells in generating the neural stem cells of the present invention.

As is mentioned hereinabove, and according to the teachings of the present invention, generation of neural stem cells from stem cells requires exposure of the stem cells to GATA6 or GATA4 or active portions or modificants thereof.

GATA6 is a 449 amino acids, 45386 Dalton, nuclear localized protein which includes two GATA-type Zinc fingers. GATA6 is expressed in the ICM Koutsouralis at al. Development 126. 723. 1999) and in the visceral endoderm of the pregastrulation embryo (Monissey et al. Genes & Dev. 12. 3579. 1998). Studies have shown that GATA6 is present in the lateral mesoderm of somitic stage embryos and later in heart, gut, and gut-derived tissues in humans (Molkentin. J. Biol. Chem. 275. 38949. 2000). GATA6 is a transcriptional activator which is thought to be important in regulation of terminal differentiation and/or proliferation of endoderm derivatives (for further description of this protein please see, http://www.rzpd.de/cgi-bin/cards/carddisp?GATA6). To date, involvement of this protein in differentiation of neural tissue has not been shown or suggested.

GATA4 is a 442 amino acids, 44626 Dalton nuclear localized protein which includes two GATA-type Zinc fingers. GATA4 is transcriptional activator which binds to the consensus sequence 5′-agatag-3′ to regulate a set of cardiac-specific genes and play a crucial role in cardiogenesis (for further description of this protein please see, http://www.rzpd.de/cgi-bin/cards/carddisp?GATA4). To date, involvement of this protein in differentiation of neural tissue has not been shown or suggested.

Several approaches can be utilized to expose such stem cells to GATA6 or GATA4 or active portions or modificants thereof.

For example, stem cells can be cultured in the presence of at least one of these proteins formulated in a manner which is suitable for facilitating intracellular transport or diffusion of these proteins. In such cases, GATA6 or GATA4 or active portions or modificants thereof can be chemically synthesized using well known synthesis techniques such as exclusive solid phase synthesis, partial solid phase synthesis methods, fragment condensation, classical solution synthesis. These methods are preferably used when the molecule is relatively short (<10 KDa) such as the case for active portions.

Alternatively, GATA6 or GATA4 or active portions or modificants thereof can be recombinantly synthesized using prokaryotic or eukaryotic expression vectors.

A variety of prokaryotic or eukaryotic cells can be used as host-expression systems to express the polypeptide coding sequence. One of ordinary skill in the art would be more than capable of constructing and utilizing various expression vectors for the purpose of expressing and isolating GATA6 or GATA4 or active portions or modificants thereof.

Expression can be effected in, for example, microorganisms, such as bacteria transformed with a recombinant bacteriophage DNA, plasmid DNA or cosmid DNA expression vector containing the coding sequence of GATA6 or GATA4 or active portions or modificants thereof; yeast transformed with recombinant yeast expression vectors containing the coding sequence; plant cell systems infected with recombinant virus expression vectors (e.g., cauliflower mosaic virus, CaMV; tobacco mosaic virus, TMV) or transformed with recombinant plasmid expression vectors, such as Ti plasmid, containing the coding sequence. Mammalian and insect expression systems can also be used to express the polypeptide of the present invention. Bacterial systems are preferably used to produce recombinant proteins since they enable a high production volume at low cost.

In bacterial systems, a number of expression vectors can be advantageously selected depending upon the use intended for the polypeptide expressed. For example, when large quantities of GATA6 or GATA4 or active portions or modificants thereof are desired, vectors that direct the expression of high levels of the protein product, possibly as a fusion with a hydrophobic signal sequence, which directs the expressed product into the periplasm of the bacteria or the culture medium where the protein product is readily purified may be desired. Certain fusion protein engineered with a specific cleavage site to aid in recovery of the conjugate may also be desirable. Such vectors adaptable to such manipulation include, but are not limited to, the pET series of E. coli expression vectors [Studier et al. (1990) Methods in Enzymol. 185:60-89).

In yeast, a number of vectors containing constitutive or inducible promoters can be used, as disclosed in U.S. Pat. No. 5,932,447. Alternatively, vectors can be used which promote integration of foreign DNA sequences into the yeast chromosome.

In cases where plant expression vectors are used, the expression of the above described coding sequence can be driven by a number of promoters. For example, viral promoters such as the 35S RNA and 19S RNA promoters of CaMV [Brisson et al. (1984) Nature 310:511-514], or the coat protein promoter to TMV [Takamatsu et al. (1987) EMBO J. 6:307-311] can be used. Alternatively, plant promoters such as the small subunit of RUBISCO [Coruzzi et al. (1984) EMBO J. 3:1671-1680 and Brogli et al., (1984) Science 224:838-843] or heat shock promoters, e.g., soybean hsp17.5-E or hsp17.3-B [Gurley et al. (1986) Mol. Cell. Biol. 6:559-565] can be used. These constructs can be introduced into plant cells using Ti plasmid, Ri plasmid, plant viral vectors, direct DNA transformation, microinjection, electroporation and other techniques well known to the skilled artisan. See, for example, Weissbach & Weissbach, 1988, Methods for Plant Molecular Biology, Academic Press, NY, Section VIII, pp 421-463.

Other expression systems such as insects and mammalian host cell systems, which are well known in the art can also be used by the present invention.

Proteins of the present invention can be purified using a variety of standard protein purification techniques, such as, but not limited to, affinity chromatography, ion exchange chromatography, filtration, electrophoresis, hydrophobic interaction chromatography, gel filtration chromatography, reverse phase chromatography, concanavalin A chromatography, chromatofocusing and differential solubilization.

Proteins of the present invention are preferably retrieved in “substantially pure” form. As used herein, “substantially pure” refers to a purity that allows for the effective use of the protein in applications described below.

Intracellular transport of the synthesized protein can be facilitated, for example, by associating it with carriers including liposomes, virosomes, microspheres and microcapsules formed of synthetic and/or natural polymers (e.g., lipids). Methods of preparing such carriers are known to the skilled in the art and include solvent evaporation, solvent casting, spray drying and solvent extension. Examples of positively charged lipids which may be used to produce cationic liposomes include the aminolipid dioleoyl phosphatidyl ethanolamine (PE), which possesses a positively charged primary amino head group; phosphatidylcholine (PC), which possess positively charged head groups that are not primary amines; and N[1-(2,3-dioleyloxy)propyl]-N,N,N-triethylammonium (“DOTMA,” see Feigner et al., Proc. Natl. Acad. Sci. USA, 84:7413-7417 (1987); Felgner et al., Nature, 337:387-388 (1989); Felgner, Advanced Drug Delivery Reviews, 5:163-187 (1990)). and proteins and peptides.

Liposomes can be generated by methods well known in the art such as those reported by Kim et al., Biochem. Biophys. Acta, 728:339-348 (1983); Liu et al., Biochem. Biophys. Acta, 1104:95-101 (1992); and Lee et al., Biochem. Biophys. Acta, 1103:185-197 (1992); Wang et al., Biochem., 28:9508-9514 (1989).

Non-vesicle carriers can also be utilized by the present invention. Such carriers include proteins or chemicals which can be associated or chemically attached to GATA6 or GATA4 proteins or active portions or modificants thereof [see for example, Namiki et al. Biochem Biophys Res Commun. 2003 Jun. 6; 305(3):592-7; Belting, Trends Biochem Sci. 2003 March; 28(3): 145-51 and Taylor et al. Electrophoresis 2003 May; 24(9):1331-7].

Although time consuming and tedious to perform, various microinjection and other mechanical approaches which are well known in the art can also be used to deliver the GATA6 or GATA4 proteins or active portions or modificants thereof into stem cells.

Alternatively and preferably, exposure of stem cells to GATA6 or GATA4 proteins or active portions or modificants thereof is effected via expression of these proteins within transformed stem cells. Information which can be used identify and design active portions or modificants of these protein can be found in the Weizmann GeneCards data at http://www.rzpd.de/cards/index.html or specifically at http://www.rzpd.de/cgi-bin/cards/carddisp?GATA6 for GATA6 and http://www.rzpd.de/cgi-bin/cards/carddisp?GATA4 for GATA4).

Since stem cells are oftentimes cultured using a feeder cell layer, the GATA6 or GATA4 proteins or active portions or modificants thereof can alternatively be expressed by transformed feeder cells. Feeder cells are available from a number of sources, see, for example, http://www.atcc.org/Products/FeederCells.cfn. Expression of GATA6 or GATA4 proteins or active portions or modificants thereof in feeder cells is preferably effected in a manner which allows secretion of the expressed proteins to the culture medium. Secretion of feeder cell expressed proteins can be effected by utilizing an expression vector or system suitable which is capable of directing secretion of expressed protein. For example, the Saccharomyces cerevisiae invertase (SUC2) signal sequence can be utilized in a mammalian expression system (see examples below) in order to direct secretion of translationally fused protein [for further detail see, for example, Farell et al., Proteins. 2000 Oct. 1; 41(1):144-53 and Lo et al., Protein Eng. 1998 June; 11(6):495-500]. It will be appreciated that since the stem cells are cultured in contact with the feeder layer and in a proliferative state, it is conceivable that proteins secreted by the feeder cells into the medium would be taken up by the stem cells and the desired effect would be achieved.

Introducing expression vectors into mammalian cells can be effected using any one of several well known techniques including direct DNA uptake techniques, and virus or liposome mediated transformation (for further detail see, for example, “Methods in Enzymology” Vol. 1-317, Academic Press).

To express these proteins in mammalian cells (i.e., the stem cells or feeder cells), a polynucleotide sequence encoding the GATA6 or GATA4 proteins (GenBank Accession numbers provided in the Examples section below) or active portions or modificants thereof can be ligated into a nucleic acid construct which is suitable for mammalian expression. Examples of suitable vectors include, but are not limited to, pcDNA3, pcDNA3.1(+/−), pZeoSV2(+/−), pSecTag2, pDisplay, pEF/myc/cyto, pCMV/myc/cyto, pCR3.1, which are available from Invitrogen, pCI which is available from Promega, pBK-RSV and pBK-CMV which are available from Stratagene, pTRES which is available from Clontech, and their derivatives and modificants.

Any of the promoter and/or regulatory sequences included in the mammalian expression vectors described above can be utilized to direct the transcription of a polynucleotide sequence encoding GATA6 or GATA4 or portions or modificants thereof. However, since such vectors are readily amenable to sequence modifications via standard recombinant techniques, alternative or additional regulatory elements, promoter and/or selection markers can easily be incorporated therein if needed. The Examples section which follows provides additional information relating to suitable vectors and transformation approaches.

It will be appreciated that since temporal control over expression of GATA6 and GATA4 is presently preferred, in particular when utilized in cultured stem cells, the expression system utilized by the present invention is preferably selected capable of inducible expression. Examples of inducible expression systems include the Complete Control® and the LacSwitch®II Inducible Mammalian Expression Systems available from StrataGene Inc. (USA) and the Tet-On™ Tet-Off™ Gene Expression System available from Clontech Inc. (USA).

When expressed in stem cells, the nucleic acid construct encoding GATA6 or GATA4 or portions or modificants thereof preferably includes a promoter sequence which enables transcription and subsequent translation (expression) of GATA6 or GATA4 or portions or modificants thereof in stem cells only. Examples of such promoters include but are not limited to EF1α promoter, or to the CMV enhancer—chicken β-actin promoter, described in the Examples section which follows. Since expression of GATA6 or GATA4 in stem cells is only necessary until induction of early differentiation, the promoter sequence utilized by the nucleic acid construct of the present invention is preferably only active in the transformed stem cells prior to induction of differentiation and is inactive thereafter. Examples of such promoters include but are not limited to, the Spi2A minimal promoter (Terskikh et al. Science 2000, 290:1585-1588) and the telomerase reverse transcriptase (TERT) promoter [Horikawa et al., Cancer Res. 1999 Feb. 15; 59(4):826-30]. Additional promoters which are active in, for example proliferating tumor cells may also be suitable for use with the nucleic acid construct of the present invention.

Alternatively, when applicable (e.g., cultures), an inducible promoter (e.g., the Tet-On™ Tet-Off™ promoter, see additional examples above) can also be used to control the expression of GATA6 or GATA4 in stem cells.

It will be appreciated that other controllable expression schemes are also envisioned by the present invention. For example use of a binary expression system in which the product of one expression construct regulates a promoter driving expression of GATA6 or GATA4 from another expression construct is also contemplated by the present invention. Such a binary system can employ, for example, a promoter active in differentiating cells (e.g., the tubulin III promoter) which drives the transcription of an siRNA molecule [Hutvagner and Zamore (2002) Curr. Opin. Genetics and Development 12:225-232] which targets GATA6 or GATA4 transcripts transcribed from the second expression construct using a constitutive promoter (e.g., CMV). Using such an approach leads to siRNA silencing of GATA6 or GATA4 expression in differentiating cells. Additional approaches for stem cell restricted expression of GATA6 and GATA4 can be readily formulated by one of ordinary skill in the art motivated to practice such cell type-restricted expression.

Exposure of the stem cells to GATA6 or GATA4 using any of the above described approaches is preferably effected under culturing conditions which are optimized for neural stem cells generation. As is illustrated in the Examples section which follows such conditions preferably include DMEM/F12 with 5 μg/ml insulin, 100 μg/ml transferrin, 16.1 μg/ml putrescine, 5.2 ng/ml selenite and 6.3 ng/ml progesterone (supplied as 1% N2 supplement, Gibco) (see the Examples section hereinunder for further detail). Other suitable culturing conditions would be readily apparent to one of ordinary skill in the art.

Since the neural stem cells of the present invention can be utilized in human therapy (described hereinbelow), culturing conditions minimize the use of xenocontaminants by using for example recombinant factors, culture medium and feeder cells approved for human therapeutic uses.

Culturing under such conditions is maintained for a time period sufficient for the induction of neural stem cell differentiation as is determined using various neural stem cell markers (see the Examples section hereinbelow for further detail).

As is illustrated in the Examples section which follows, the methodology described herein can be used to generate stem cell cultures in which at least 40-70% or more of the cells are neural stem cells which are capable of differentiating into neurons and glial cells.

Such neural stem cells can be easily identified via marker expression and/or morphology (see examples section for further detail) and subsequently isolated using, for example, an automated cell sorter.

Since the present method is clonal competent, once neural stem cells generated according to the teachings of the present invention are isolated, such cells can be utilized to generate dense cultures.

Neural stem cells of the present invention can be utilized in various therapeutic approaches.

Thus, according to another aspect of the present invention there is provided a method of treating a neurological disorder characterized by neural cell degeneration or loss. Examples of neurological disorders characterized by neural cell degeneration or loss include, but are not limited to, Parkinson's disease, Huntington chorea, Alzheimer's disease, brain hemorrhage and myelination disorders familial autonomic diseases, neurofibroma, neuroblastoma, pheochromocytoma, various types of dementia such as senile dementia, Creutzfeldt-Jakob disease, bovine spongiform encephalopathy, and scrapie.

The treatment method according to this aspect of the present invention is effected by administering the neural stem cells of the present invention to a subject diagnosed with the neurological disorder thereby treating the neurological disorder characterized by neural cell degeneration or loss.

Such administration can be effected by systemic or direct injection of the neural stem cells to the subject using, for example, a syringe or catheter designed or adapted for cellular injection.

Since the neural stem cells of the present invention are capable of efficiently targeting to and exclusively colonizing CNS tissues (see the Examples section which follows for further detail), systemic administration thereof would lead to desired therapeutic effect and thus in sharp contrast to therapeutic approaches which utilize prior art stem cell preparations, systemic administration of the neural stem cells of the present invention can lead to effective treatment of the disorder.

It will be appreciated that when the method described above is practiced using neural stem cells generated from adult stem cells, the adult stem cells can be harvested from the individual to be treated thus minimizing the possibility of an immune reaction or graft versus host disease.

Although the above described therapeutic approach is presently preferred, the present invention envisages alternative therapeutic treatments which utilize an in-vivo approach.

Since nucleic acid constructs which express GATA6 or GATA4 exclusively in stem cells can be constructed (see description hereinabove) and since the neural stem cell generated according to the teachings of the present invention target to and exclusively colonize CNS tissues, in vivo nucleic acid transfer (i.e., gene therapy) into cells including stem cells would lead to the desired therapeutic effect.

Currently preferred in vivo nucleic acid transfer techniques include transfection with viral or non-viral constructs, such as adenovirus, lentivirus, Herpes simplex I virus, or adeno-associated virus (AAV) and lipid-based systems. Useful lipids for lipid-mediated transfer of the gene are, for example, DOTMA, DOPE, and DC-Chol [Tonkinson et al., Cancer Investigation, 14(1): 54-65 (1996)]. The most preferred constructs for use in gene therapy are viruses, most preferably adenoviruses, AAV, lentiviruses, or retroviruses. A viral construct such as a retroviral construct includes at least one transcriptional promoter/enhancer or locus-defining element(s), or other elements that control gene expression by other means such as alternate splicing, nuclear RNA export, or post-translational modification of messenger. Such vector constructs also include a packaging signal, long terminal repeats (LTRs) or portions thereof, and positive and negative strand primer binding sites appropriate to the virus used, unless it is already present in the viral construct. The construct may include a signal that directs polyadenylation, as well as one or more restriction sites and a translation termination sequence. By way of example, such constructs will typically include a 5′ LTR, a tRNA binding site, a packaging signal, an origin of second-strand DNA synthesis, and a 3′ LTR or a portion thereof. Other vectors can be used that are non-viral, such as cationic lipids, polylysine, and dendrimers.

Thus, the present invention provides a novel and readily applicable method of generating high quantities of transplantation-competent and differentiation capable neural stem cells which can be used in a variety of therapeutic approaches.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

### Example 1

### Materials and Methods

Culture Conditions

All ES cell lines (Table 1) were grown on a feeder layer of X-irradiated mouse embryo fibroblasts in ES medium which included DMEM supplemented with 15% fetal calf serum [FCS], 1% glutamine, 0.1 mM P-mercapto-ethanol, 20 g/ml Heparin, penicillin, streptomycin and a previously tittered dilution of LIF as the supernatant fibroblast transfected by a LIF plasmid. The medium was changed daily (Chen et al. Oncogene 19, 3750-3756; 2000).

Embryoid bodies: ES cells cultured on feeder cells were removed via trypsinization and subsequently twice adhered to gelatinized tissue culture dishes for 50 minutes in the presence of EB medium, which is identical to the ES medium described above, but is devoid of LIF. Following removal of the feeder cells; 1-2×10⁶ ES cells were plated on a 9 cm tissue culture plate and the plate was incubated overnight at 37° C. under 5% CO₂. Primary ES cell aggregates that formed during this time period were removed using a pipette and transferred to bacteriological (Sterilin) tissue culture plates. Embryoid bodies were grown with daily medium change for five days or a week, till both epithelial layers developed and cavitation took place.

Neural derivatives: GATA4 or GATA6 transfected ES cells (described below) originally grown as mass cultures on tissue culture dishes in ES medium were trypsinized and 10³ cells were deposited on glass coverslips that had been coated with a solution of 20 μg/ml poly-D-lysine and 250 μg/ml fibronectin and placed into each well of 12-well plates. The cells were grown on the coverslips in the presence of a defined differentiation medium which included DMEM/F12 with 5 μg/ml insulin, 100 μg/ml transferrin, 16.1 μg/1 ml putrescine, 5.2 ng/ml selenite and 6.3 ng/ml progesterone (supplied as 1% N2 supplement, Gibco).

TABLE 1
Cell lines

| Designation | Origin | Characteristics | Reference |
|---|---|---|---|
| R1 | 129 ES | Wild type | Nagy et al. Proc. Natl. |
| | cell line | | Acad. Sci. U.S.A. 90, |
| | | | 8424-8428 (1993). |
| AB2.2 | 129 ES | ″ | McMahon and Bradley |
| | cell line | | Cell 62, 1073-1085 |
| | | | (1990) |
| ROSA 11 | AB2.2 | carries βgeo reporter | Friedrich and Soriano |
| (R11) | | and resistance | Genes Dev 5, 1513- |
| | | | 1523 (1991) |
| 1C6 | R11 | Carries truncated | Chen et al. Oncogene |
| | | Fgfr2c cDNA | 19, 3750-3756 (2000) |
| Lamc1+/− | R1 | Heterozygous mutant | Smyth et al. J. Cell |
| | | of Lamc1 | Biol. 144, 151-160 |
| | | | (1999) |
| Lamc1−/− | ″ | Homozygous mutant | Smyth et al. J. Cell |
| | | of Lamc1 | Biol. 144, 151-160 |
| | | | (1999) |

DNA Constructs and Transfection into ES Cells

GATA4 (NM_{—}08092, mouse; NM_{—}002052 human) or GATA6 (NM_{—}128828, mouse; NM_{—}131557, human) cDNA were ligated into the pCAGI (Fujikura, J. et al. Genes Dev 16, 784-789. 2002) or pBOS (Mizushima and Nagata Nucleic Acids Res 18, 5322. 1990) vectors. The pCAGI vector included the CMV enhancer and the chicken β-actin promoter (Fujikura, J. et al. Ibid) and carried a CpG island, which protected the inserted coding sequence from transcriptional silencing. The pCAGI vector also included an IRES-puromycin resistance gene (Fujikura, J. et al. Genes Dev 16, 784-789. 2002). The pBOS vector included the EF1-αpromoter (Mizushima and Nagata Nucleic Acids Res. 8. 5322. 1990).

These constructs (10 μg/each) were electroporated into 107 ES cells together with 2 μg of a puromycine resistance vector utilizing the PGK1 promoter (Adra et al., Gene, 60. 65. 1987). The cells were distributed onto three 9 cm tissue culture plates and puromycine was added to the ES cell medium on the second day of culturing. Clones became visible 4 to 5 days following transfection and were picked into 24 well plates between day 6 and 8. Several million cells of the transfected clones were then frozen in the presence of 10% mercaptoethanol and stored in liquid nitrogen.

Immunofluorescence

Cells grown on coverslips in the defined differentiation medium described above were incubated in 5% FCS for 30 minutes at room temperature and the axonal network of these cells was immunostained using an anti-tubulin beta III monoclonal antibody (Tuj-1, Covance Research Products, Berkeley, Calif., diluted 1:400). Astrocytes were stained with an anti glial fibrillary acid protein (GFAP) monoclonal antibody conjugated to a Cy3 fluorescent tag (Sigma, 1:400). Neuron-specific neuronal enolase (Serotec) and the glial cell specific S-100 antibodies were also used. After washing the cell preparation with PBS (3 times, 5 minutes each wash), an FITC- or Cy3-conjugated goat anti-mouse IgG or IgM secondary antibody (Jackson ImmunoResearch Lab. Inc, 1:400 in PBS) was added to the cells for 1 h at room temperature, following which the cells were washed with PBS 3 times for 5 minutes and mounted in Mowiol. Samples were examined with an Olympus IX-70 FLA microscope under UV-light fluorescence. Photographs were made with a microscope-mounted DVC digital camera and were processed using Photoshop 6.0 (Adobe Inc.).

Transplantation Assays

GATA6 transfected R11 cells and control non-transfected R11 cells (see Table 1), were injected subcutaneously into 129/SvPas mice (Simpson et al. Nat. Genet. 16. 19. 1997). The non-transfected R11 cells grew into 1 cm diameter teratomas within three weeks, whereas the GATA6 transfected R11 cells did not exhibit any growth over a period of four months.

Colonization of the brain: 5×10⁴ GATA6 transfected R11 cells were injected into the lateral ventricle of newborn non-inbred CD1 mice, using a Hamilton syringe. The mice were sacrificed 35 days following transplantation, subjected to intracranial perfusion with 1% paraformaldehyde and the brains were removed and sectioned into 25 μm cryo-sections. The transplanted cells were detected via β-galactosidase staining.

Contribution to early neurogenesis: E3.5 C57BL/6 mouse blastocyst (E3.5-3.5 days post coutum) was micro-injected with approximately ten GATA6 transfected R11 cells, or with approximately ten control R11 cells using an Olympus microinjection apparatus. The blastocysts were transplanted into the uterus of 2.5 months old CD1 female mice which was mated to a vasectomized male 2.5 days before transplantation. The embryos were isolated at the E11.5 stage, pre-fixed in 0.5% glutaraldehyde and stained to detect β-galactosidase activity. The stained embryos were photographed using an Olympus stereomicroscope with a CCD camera. The β-galactosidase stained embryos were then postfixed in 4% paraformaldehyde for 2 hours, dehydrated through an EtOH series (10 minutes each stage) and embedded in paraffin. Five μm sections were stained with Eosin and photographed using a Nikon DXM1200 microscope attached to a CCD camera.

### Results

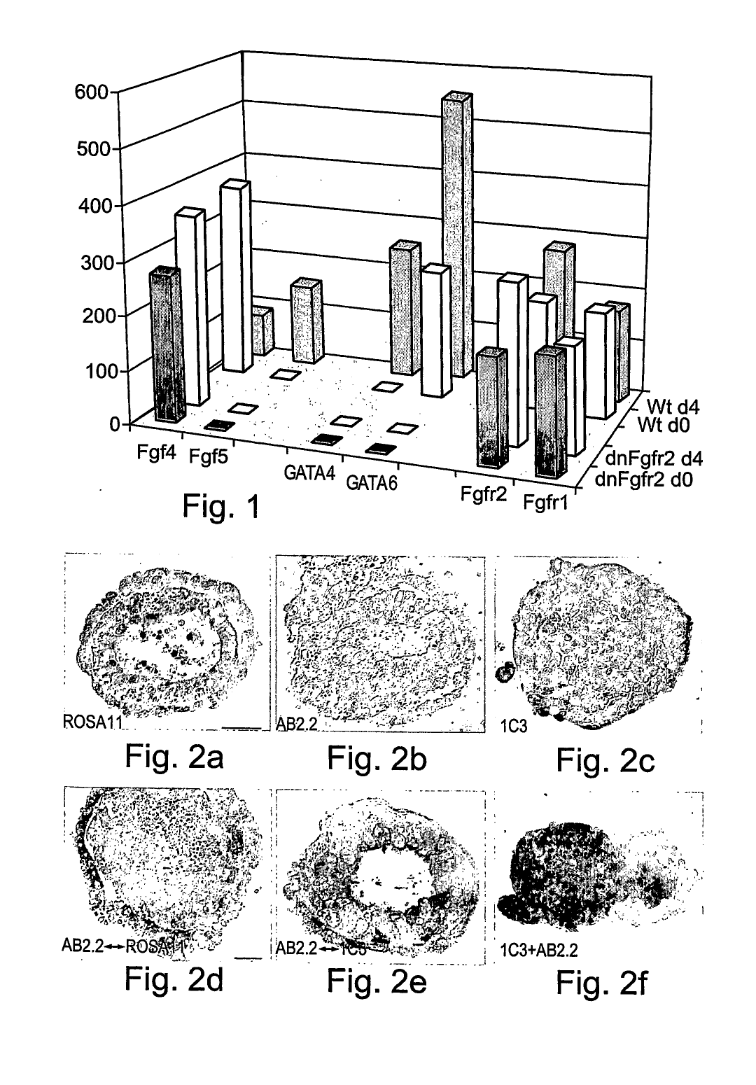

FGF signaling is required for embryoid body differentiation DNA experiments conducted using Affymetrix chips (U74 http://affymetrix.com) illustrated that ES cells and early embryoid bodies express multiple FGF receptor (FIG. 1). To assess the importance of FGF signaling in early embryogenesis, the present study applied a dominant negative approach using Fgfr2IIIc cDNA (M_{—}010207) truncated ten nucleotides downstream of the trans-membrane domain to modify the differentiation of ES cell derived embryoid bodies. ES cells, expressing truncated Fgfr2, did not differentiate and cavitate, suggesting that FGF signaling is required for the differentiation of the visceral endoderm and the primitive ectoderm, the cell layers comprising the embryoid body and the pre-gastrulation mammalian embryo (Chen et al. Oncogene 19, 3750-3756 2000).

FGF Signaling and Basement Membrane Assembly are Connected

Embryoid bodies were grown from a mixture of normal ES cells and ES cells expressing truncated Fgfr2 cDNA in order to understand the role of FGF signaling in early embryonic development. Since the cells utilized express a β-galactosidase reporter, it was possible to assess whether wild type cells can rescue the mutant defect. Under such a chimeric environment, the mutant cells expressing the truncated Fgfr2 cDNA contributed to both layers of the embryoid body (FIG. 2). Since no hypothetical differentiation factor could be found in the culture supernatant, it was assumed that FGF signaling is in some ways connected to processes involving the extra cellular matrix (ECM), which is in intimate contact with the adjacent cell layers of the embryoid body.

To test this hypothesis, the presence of basement membrane (BM) proteins was investigated by analyzing protein and mRNA levels of laminin and collagen-type-IV isotypes, which constitute the network forming elements of the BM.

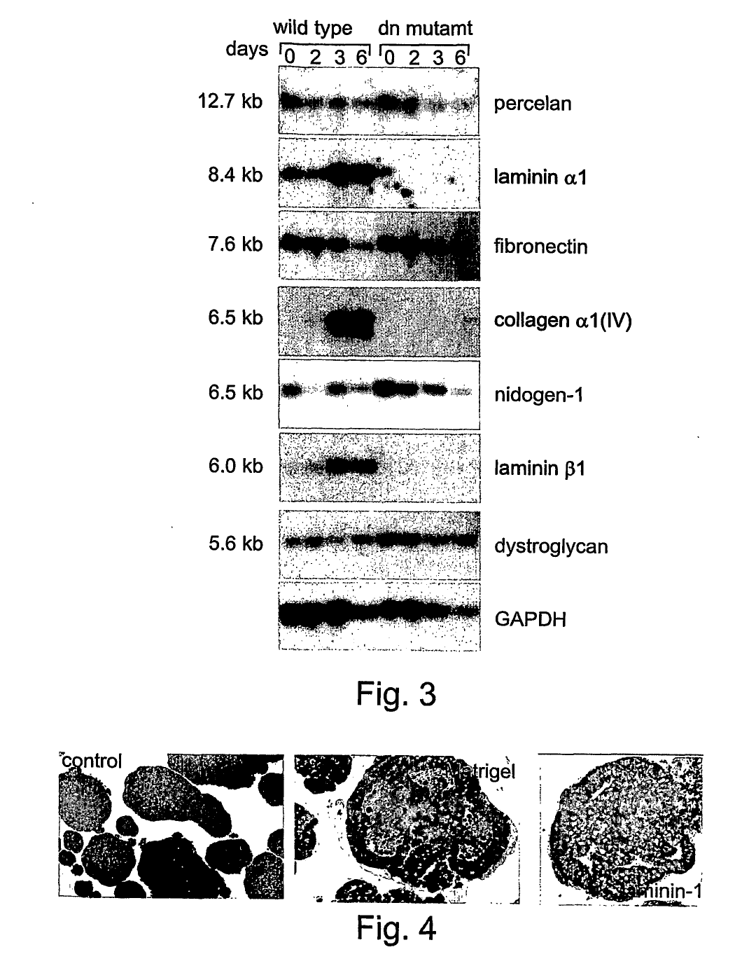

As is shown in FIG. 3 dnFGFR abrogated laminin-α1 and β1, as well as collagen IV1α synthesis, without interfering with the synthesis of other BM proteins. Additional experiments illustrated that laminin-1 (the active heterotriplex of laminin-α1, β1 and γ1) could partially rescue embryoid body differentiation of dnFGFR expressing ES cells (FIG. 4). These results have led to the hypothesis that the primitive endoderm or hypoblast produces the basement membrane, which in addition to its structural role, contributes to the epithelial transition of ES cells into the epiblast (Li, X. et al. J. Cell. Biol. 153, 811-822.2001; Ekblom et al. Matrix Biol 22, 35-47.2003).

The ICM-to-epiblast Transition is Mediated by the Sub-Endodermal BM

The above described rescue experiments suggested that sub-endodermal BM represents the default signal required for epiblast differentiation (Li et al. J. Cell. Biol. 153.811.2001), which is a prerequisite of gastrulation. Epiblast differentiation transforms the three-dimensional aggregate of the ICM, to the columnar epithelium of the epiblast, a process similar to that observed in embryoid body differentiation (Coucouvanis et al. Cell 83, 279-287.1995). Since endoderm-like cells were needed in order to provide evidence for the role of the BM in the ICM-epiblast interaction, experiments utilizing GATA6 and GATA4 were conducted. GATA6 and GATA4 are Zn-finger transcription factors that are required for endoderm and heart development (Morrisey et al. J Biol Chem 272, 8515-8524). GATA6, which is expressed earlier in development, is transcribed in the ICM and induces GATA4 (Morrissey et al. Genes & Dev. 12.3579.1998).

GATA4 and GATA6 are activated by FGF signaling and induce endoderm differentiation in ES cells in vitro, as well as in the ICM of the blastocyst in vivo. The endoderm activated by GATA6 or 4 expresses BM proteins on its basal surface. The subendodermal BM represents the default signal for epiblast differentiation which leads to cytoskeletal transformation of ES cells into columnar ectoderm (as shown in FIG. 6) and leads to formation of the polar columnar epithelium of the pre-gastrulation epiblast. Relevant to these cytoskeletal rearrangements is that laminin-1 is required for cyst or tube formation controlled by small GTPases, such as Rho and Rac (O'Brien et al. Nat Cell Biol 3, 831-838. 2001).

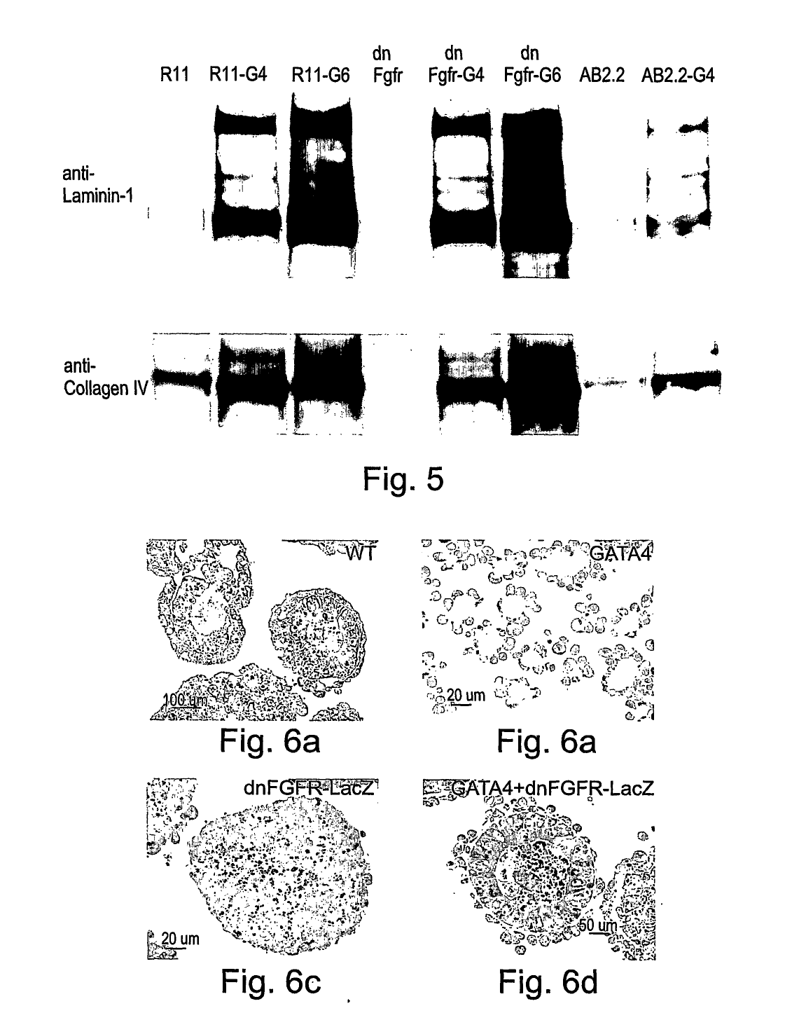

Thus, to induce endoderm differentiation, ES cell lines were transfected with expression constructs which included the GATA6 or GATA4 coding sequences positioned under the control of the CMV enhancer and chicken beta-actin promoter (PCAGI) or the EF1a sequence of the pBOS vector (see description above). Both wild type and dnFGFR mutant GATA-transformed ES cells expressed large amounts of laminin-1 and Collagen type IV (FIG. 5) and a number of markers characteristic for the endoderm (hypoblast lineage). When these cells were added to Fgfr-mutant ES cells, complete embryoid body differentiation was obtained (FIG. 6).

GATA6 Induces a Common Hypoblast and Neuroectoderm Precursor

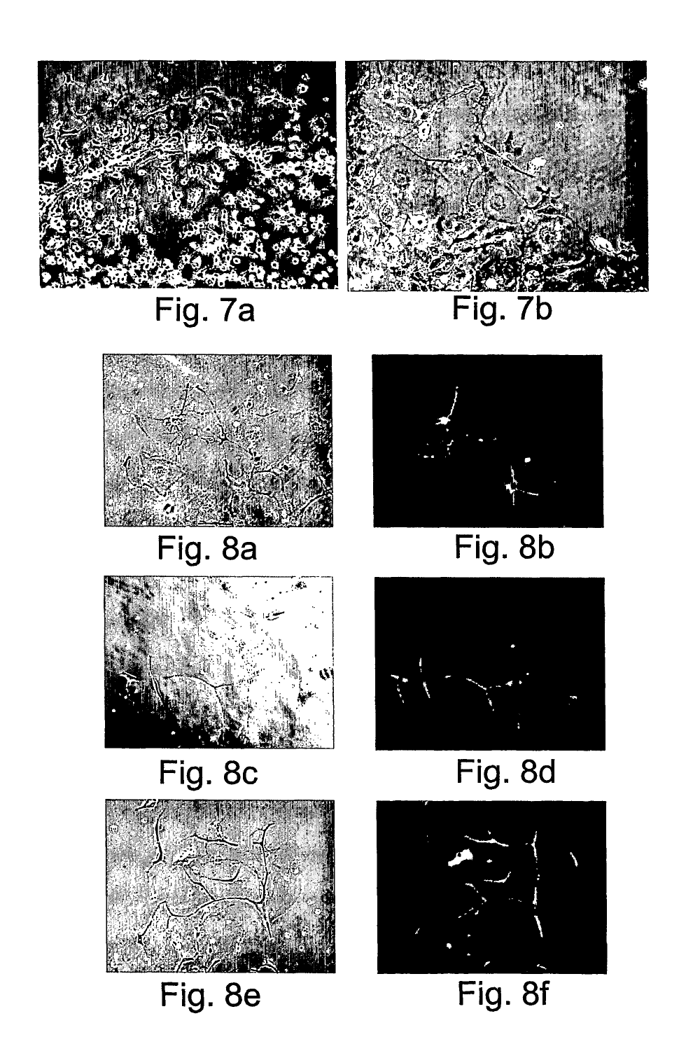

Closer inspection of GATA6 transformed ES cell colonies lead to an unexpected and astounding observation. GATA6-transformed cells, which were loosely grouped small round cells, were surrounded by cells characterized by long thin extensions typical of early neurons or glia cells (FIG. 7). Several independent transfections all included cells which exhibited such neuron-like morphology. Since the great majority of the clones were derived from a single individual founder cell, we concluded that both cell types, the neuron-like and the endoderm-like cell, develop from a single common precursor.

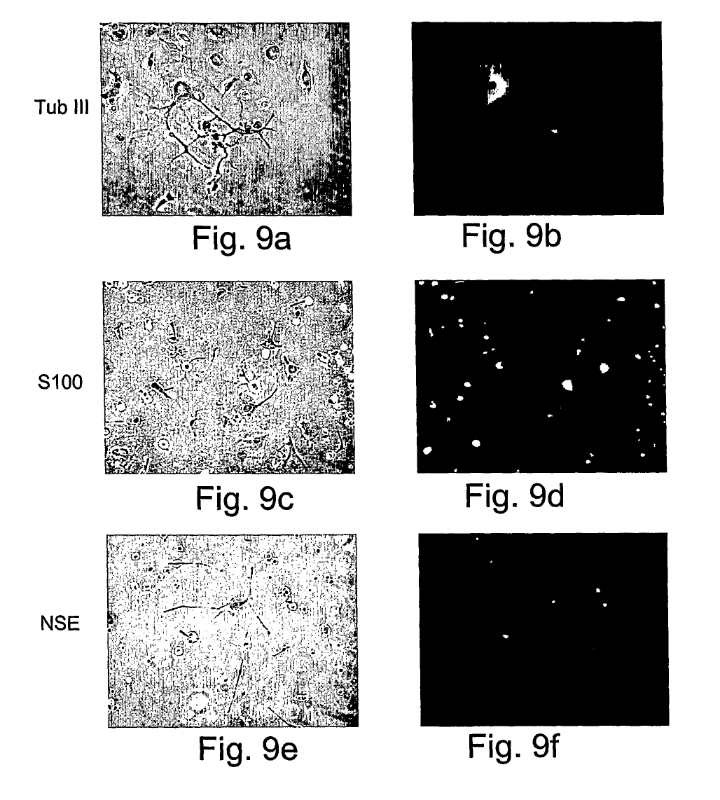

Culturing the neuron-like cells described above in a serum-free medium used for growing neurospheres (2,17) resulted in a larger proportion of GATA6-transformed ES cells which exhibited neural morphology. In such cultures 40-70% of the cells stained with neuron specific tubulin (FIG. 8) or neuron specific enolase which detect neurons or with S100 which detects glia cells (FIG. 9) thus confirming that GATA6 or GATA4 transfected ES cells develop into early neurons and glia cells.

In order to assess whether these cells can contribute to CNS development a number of transplantation experiments were performed.

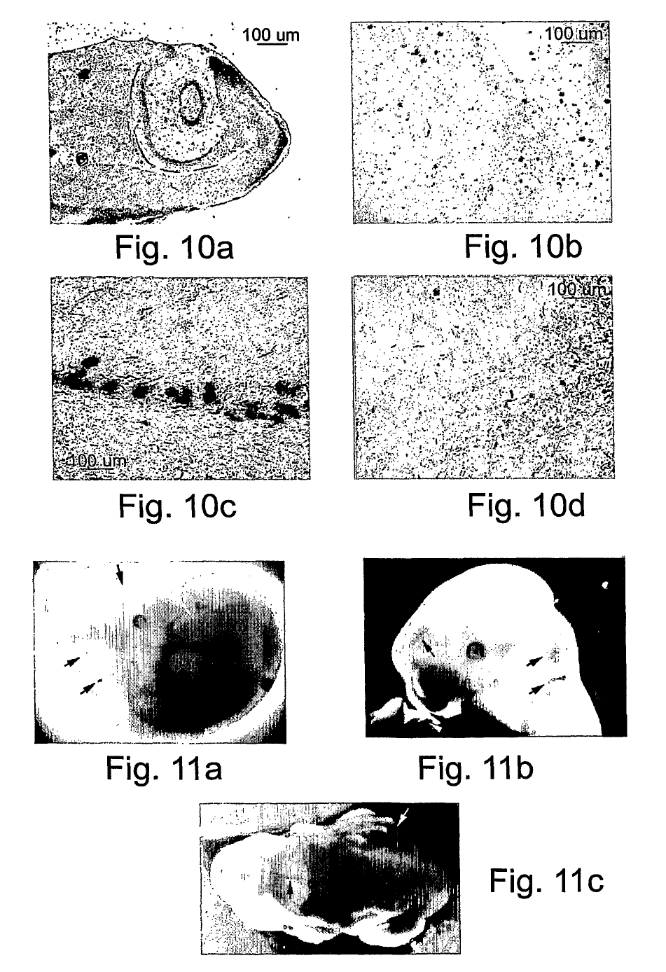

In contrast to wild type ES cells, ES cells expressing GATA6 or GATA4 do not form tumors or teratomas in congenic 129 mouse strains following more than four months of observation. Using a β-galactosidase reporter assay, transplanted GATA6-transformed cells and their derivatives could be detected close to the hippocampus (FIG. 10A) in a channel-like structure interconnecting with the ventricle (FIGS. 10B and C) and also in deeper brain regions, close to the C3 area (FIGS. 10B and C).

In order to simulate neural induction, GATA6-transfected ES cells were microinjected into blastocysts, which were transplanted into pseudopregnant female mice. At the E11.5 stage, β-galactosidase expressing cells could be detected in the prospective hind, mid and fore brain (FIGS. 11A and B), as well as in the sulcus limitans of the midbrain and in the floor plate of the spinal cord (FIG. 11C). A large number of β-galactosidase positive cells colonized the yolk sack, but no GATA6 transformed cells could be detected in any other sites. Compared to pluripotent wild type ES cells, which colonize most organs and tissues, the GATA6-transform stem cell is more specific, and is restricted to endoderm hypoblast) and neuroectoderm related tissues.

These important findings clearly indicate that the present methodology can be utilized to replace the presently available complex and empirical methodologies used to generate neural stem cells suitable for treatment of disorders characterized by degeneration or loss of neural cells.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications and GenBank Accession numbers mentioned in this specification are herein incorporated in their entirety by reference into the specification, to the same extent as if each individual publication, patent or patent application or GenBank Accession number was specifically and individually indicated to be incorporated herein by reference. In addition, citation or identification of any reference in this application shall not be construed as an admission that such reference is available as prior art to the present invention.

What is claimed is:

1. A stem cell culture comprising stem cells transformed to express GATA6 or GATA4 or active portions or modificants thereof.

2. The stem cell culture of claim 1, wherein the stem cells are embryonic stem cells.

3. The stem cell culture of claim 2, wherein said embryonic stem cells are derived from an inner cell mass (ICM) layer of an embryo.

4. The stem cell culture of claim 3, wherein said embryo is a human embryo.

5. A stem cell expressing GATA6 or GATA4 or active portions or modificants thereof.

6. The stem cell of claim 5, wherein the stem cell is characterized by expression of at least one protein selected from the group consisting of nestin, class III β-tubulin, neural specific enolase, S-100 and glial specific acidic fibrillary protein (GFAP).

7. The stem cell of claim 5, wherein the stem cell is characterized by a neuron-like morphology.

8. The stem cell of claim 5, wherein the stem cell is an embryonic stem cell.

9. The stem cell of claim 5, wherein the stem cell comprises a polynucleotide sequence encoding said GATA6 or said GATA4 or active portions or modificants thereof.

10. A nucleic acid construct comprising a polynucleotide sequence encoding a GATA6 or a GATA4 or active portions or modificants thereof.

11. The nucleic acid construct of claim 10, further comprising at least one promoter sequence being for directing transcription of said polynucleotide sequence in stem cells.

12. The nucleic acid construct of claim 11, wherein said at least one promoter sequence is not active in differentiated cells.

13. The nucleic acid construct of claim 11, wherein said at least one promoter is selected from the group consisting of Spi2A minimal promoter and the hTERT promoter.

14. A method of generating neural stem cells comprising exposing stem cells to GATA6 or GATA4 or active portions or modificants thereof thereby generating neural stem cells.

15. The method of claim 14, wherein said exposing is effected for a time period sufficient for inducing expression in said stem cells of at least one protein selected from the group consisting of nestin, class III β-tubulin, neural specific enolase, S-100 and glial specific acidic fibrillary protein (GFAP).

16. The method of claim 14, wherein said exposing is effected by transforming said stem cells with a nucleic acid construct capable of expressing said GATA6 or said GATA4 or active portions or modificants thereof in said stem cells.

17. The method of claim 14, wherein said exposing is effected by culturing said stem cells on a feeder cell layer expressing said GATA6 or said GATA4 or active portions or modificants thereof.

18. The method of claim 17, wherein said feeder cell layer is capable of secreting said GATA6 or said GATA4 or active portions or modificants thereof expressed thereby.

19. The method of claim 14, wherein said stem cells are embryonic stem cells.

20. The method of claim 19, wherein said embryonic stem cells are derived from an inner cell mass (ICM) layer of an embryo.

21. The method of claim 20, wherein said embryo is a human embryo.

22. The method of claim 14, wherein said stem cells are cultured in a medium including DMEM/F12 with 3-7 μg/ml insulin, 80-120 μg/ml transferrin, 10-18 μg/ml putrescine, 3-7 ng/ml selenite and 3-7 ng/ml progesterone.

23. The method of claim 14, wherein said exposing is effected by culturing said stem cells in a culture medium supplemented with said GATA6 or said GATA4 or active portions or modificants thereof.

24. A feeder cell population comprising feeder cells transformed to express GATA6 or GATA4 or active portions or modificants thereof.

25. The feeder cell population of claim 24, wherein said feeder cells are fibroblasts.

26. The feeder cell population of claim 24, wherein said feeder cells comprise a nucleic acid construct including a polynucleotide sequence encoding said GATA6 or said GATA4 or active portions or modificants thereof.

27. A composition-of-matter comprising GATA6 or GATA4 or active portions or modificants thereof associated with a carrier suitable for directing intracellular delivery of said GATA6 or said GATA4 or active portions or modificants thereof.

28. The composition-of-matter of claim 27, wherein said carrier is a lipid carrier.

29. The composition-of-matter of claim 27, wherein said carrier is a protein carrier.

30. A method of treating a neurological disorder characterized by neural cell degeneration or loss, the method comprising:
(a) administering stem cells to a subject diagnosed with the neurological disorder; and
(b) prior to, concomitant with or following administration, exposing said stem cells to GATA6 or GATA4 or active portions or modificants thereof to thereby treat the neurological disorder characterized by neural cell degeneration or loss.

31. The method of claim 30, wherein said exposing is effected for a time period sufficient for inducing expression in said stem cells of at least one protein selected from the group consisting of nestin, class III β-tubulin, neural specific enolase, S-100 and glial specific acidic fibrillary protein (GFAP).

32. The method of claim 30, wherein said exposing is effected by transforming said stem cells with a nucleic acid construct capable of expressing said GATA6 or said GATA4 or active portions or modificants thereof in said stem cells.

33. The method of claim 30, wherein said stem cells are embryonic stem cells.

34. The method of claim 33, wherein said embryonic stem cells are derived from an inner cell mass (ICM) layer of an embryo.

35. The method of claim 34, wherein said embryo is a human embryo.

36. The method of claim 30, wherein said exposing is effected by co-injecting said stem cells along with said GATA6 or said GATA4 or active portions or modificants thereof.

37. A method of treating a neurological disorder characterized by neural cell degeneration or loss, the method comprising:
(a) expressing within stem cells GATA6 or GATA4 or active portions or modificants thereof to thereby generate neural stem cells; and
(b) administering said neural stem cells to a subject diagnosed with the neurological disorder thereby treating the neurological disorder characterized by neural cell degeneration or loss.

38. The method of claim 37, wherein said expressing is effected by transforming said stem cells with a nucleic acid construct capable of expressing said GATA6 or said GATA4 or active portions or modificants thereof in said stem cells.

39. The method of claim 37, wherein said stem cells are embryonic stem cells.

40. The method of claim 39, wherein said embryonic stem cells are derived from an inner cell mass (ICM) layer of an embryo.

41. The method of claim 40, wherein said embryo is a human embryo.

42. The method of claim 37, wherein said stem cells are cultured in a medium including DMEM/F12 with 3-7 μg/ml insulin, 80-120 μg/ml transferrin, 10-18 μg/ml putrescine, 3-7 ng/ml selenite and 3-7 ng/ml progesterone prior to step (b).

43. The method of claim 37, wherein step (a) is effected for a time period sufficient for inducing in said stem cells expression of at least one protein selected from the group consisting of nestin, class III β-tubulin, neural specific enolase, S-100 and glial specific acidic fibrillary protein (GFAP).
